(19) United States
(12) Patent Application Publication
(10) Pub. No.: US 2013/0245586 A1
(43) Pub. Date: Sep. 19, 2013

(54) IMMOBILIZATION SYSTEM FOR FLUID TRANSPORT TUBES

(71) Applicant: BOARD OF TRUSTEES, SOUTHERN ILLINOIS UNIVERSITY, Carbondale, IL (US)

(72) Inventor: Om Jha, Alhambra, CA (US)

(73) Assignee: Board of Trustees, Southern Illinois University, Carbondale, IL (US)

(21) Appl. No.: 13/774,812

(22) Filed: Feb. 22, 2013

Related U.S. Application Data
(60) Provisional application No. 61/602,125, filed on Feb. 23, 2012.

(51) Int. Cl. A61F 5/443

(52) U.S. Cl. CPC A61F 5/443
USPC 604/352

(57) ABSTRACT

A securement bandage for ensuring fluid transport to or from a patient is provided such that the securement bandage has a flexible sheath, and an integrated adhesion platform located at a base end of the flexible sheath. Wherein, the integrated adhesion platform is provided with a center opening.

### CROSS-REFERENCE TO RELATED APPLICATIONS

This disclosure is a Non-Provisional Patent application entitled Immobilization System for Fluid Transport Tubes. This application claims priority to and the benefit of U.S. Provisional Application No. 61/602,125, filed Feb. 23, 2012, which is hereby incorporated herein.

### BACKGROUND OF THE DISCLOSURE

1. Field of Disclosure

The present disclosure related to a means of securing a tube to a patient. More specifically, the present invention relates to a device for securing a fluid transport tube or catheter, such as a condom catheter, to a patient.

2. Background Art

There are several instances where various tubes need to be secured to a patient. The mechanisms currently available for such securement often do not properly adhere to the skin. This problem is due to a variety of reasons which may include low adhesive surface area to be applied to the body. Additionally, bandages and tapes are often not ideal for immobilizing tubes because they do not conform to the shape of the tubes. Finally, there are often general adhesion problems related to negotiating protruding body parts with uneven or unmoving surfaces. Examples of these protruding body parts might include, but are not limited to, a nose, ear, mouth or penis. Examples of tubes that can be used include, but are not limited to, a naso-gastric tube, urinary catheter, condom catheter, or other similar device.

There are several available adhesives that are insufficient in securing tubes to patients. For example, typical adhesive bandages are often linear and do not conform well to the desired surface area needed to provide sufficient adhesion. A broad range of Tegaderm™ bandages offered by 3M are capable of securing intravenous (IV) tubes on substantially flat surfaces but do not work well for tubes and protruding body parts. Some condom catheters are provided with a small area of adhesive around the circumference of the base of the catheter and in the internal tubing of the catheter. However, these condom catheters routinely fail due to fatigue in the adhesive portion. Additionally, little to no adhesion is provided to the shaft of the pediatric penis because the penis in this population is generally quite small. This adhesion problem requires the health care provider to rely on the small strip of adhesion around circumference of the base of the catheter, which is often insufficient and results in urine leakage.

Prior designs have attempted to address adhesion problems but still fall short in their effectiveness in young patients and animals. For example, U.S. Pat. No. 4,475,910 discloses a male urinal device where an adhesive layer is provided on the inside of the urinal device. This design may work well with adult patients, but does not provide a leak-free bond in pediatrics due to its inability to accommodate a small penis shaft. Additionally, U.S. Pat. No. 4,261,363 discloses a clip for retaining a flexible body fluid drain at a drainage site. This design fails to demonstrate any effectiveness for tubes and catheters used in protruding body parts, though. Finally, U.S. Pat. Nos. 4,917,112, 5,018,516, and 5,042,466 disclose different adhesive strips that may be used as bandages or holding wraps for tubes. U.S. Pat. No. 4,917,112 specifically shows a tracheal tube being secured with the disclosed bandage; however, this patent and the other disclosed adhesive strips lack applications to protruding body parts.

Thus, there still exists a need in the art for a condom catheter for the pediatric male population, especially in the case of pediatric patients with diarrhea. In pediatric patients, diarrhea is a very common yet potentially serious condition. In patients with diarrhea in an inpatient setting, the most important parameter to monitor is the effect of interventions like IV fluids through Urine Output (UO). Unfortunately, correctly measuring this simple parameter becomes very difficult as the weight of a diaper is generally used to determine the UO. Condom catheters are used on these patients to separate the stool from the urine, but these catheters frequently fail because of poor adhesion. The UO measurement is often imprecise because urine is mixed with the stool in the diaper due to the failed condom catheter. Once the condom catheter fails, a physician will typically order a Basic Metabolic Panel to assess hydration status, as well as administer IV fluid boluses. Pediatric patients may not need IV fluid boluses, and providing such fluid can place unwanted strain on their hearts. A condom catheter with reliable adhesion would prevent unneeded IV fluid boluses. Presently available condom catheters for the pediatric age group are undesirable for nurses to use because the shaft of penis does not provide adequate support to the catheter. The only means of securing the catheter to the body using current designs is an adhesive rim with an area of contact approximately 2 mm wide around the circumference of the catheter. This adhesive connection frequently fails because of the weight of catheter and corresponding urobag, secreting urine, and perspiration. Condom catheter failure represents a universal problem in every health care establishment and medical institution that works with pediatric patients.

In this example and others, securing these tubes and catheters is important for the general transport and accurate delivery of fluids to and away from the body. Thus, there is a need for an adhesive bandage or device that improves the attachment of a tube or catheter to the body, especially in the pediatric population. A bandage or device is needed that provides increased adhesion relative to the body and the tube or catheter. The device also needs to be simple to construct and easy to use. Furthermore, the tube or catheter must be secured in such a way that fluids can be extracted and introduced with minimal movement or agitation of the adhered connection during normal movement of the patient. Such securement should support extended usage time of the tube or catheter as well.

### BRIEF SUMMARY OF DISCLOSURE

An embodiment is a securement bandage for ensuring fluid transport to or from a patient, the securement bandage includes a flexible sheath, and an integrated adhesion platform located at a base end of said flexible sheath, wherein the integrated adhesion platform is provided with a center opening for the flexible sheath.

Another embodiment is a securement bandage assembly for securing a flexible sheath to a patient comprising an adhesive applicator bandage with a center opening, and a flexible sheath adhesive area along the center opening. In this embodiment, the flexible sheath is not integrated with the securement bandage. Instead, it is secured through adhesion near the center opening.

Another embodiment is a securement bandage assembly for securing a flexible sheath to a patient comprising an adhesion platform and a flexible sheath. In this embodiment, the flexible sheath is an element of the assembly.

Another embodiment is a kit for ensuring fluid transport to or from a patient, the kit comprising a securement bandage.

Another embodiment is a kit for ensuring fluid transport to or from a patient, the kit comprising a flexible sheath, and a securement bandage assembly adhesively connectable to the flexible sheath.

Another embodiment is a method of assemblying a securement bandage comprising forming a flexible sheath having a base end opening with a circular base flange extending from and about the opening and further having a distal narrowed catheter end; forming an adhesion platform having a center opening communicable with the base end opening and adhesively attached thereto; and attaching the flexible sheath to the adhesion platform.

### BRIEF DESCRIPTION OF THE DRAWINGS

For a better understanding of the present disclosure, reference may be made to the accompanying drawings in which:

FIG. 1 is an elevated perspective view of a securement bandage in a rectangular shape comprising a flexible sheath embodied by a condom catheter and an integrated adhesion platform;

FIG. 2 is a front perspective view of an optional embodiment of a securement bandage in a diamond shape comprising a flexible sheath embodied by a condom catheter and an integrated adhesion platform;

FIG. 3 is a front perspective view of another optional embodiment of a securement bandage in a rounded shape comprising a flexible sheath embodied by a condom catheter and an integrated adhesion platform;

FIG. 4 is a front perspective view of a general securement bandage without a specific shape comprising a flexible sheath and an integrated adhesion platform;

FIG. 5 is an “in use” view of a securement bandage assembly securing a flexible sheath;

FIG. 6 is an “in use” view of an applicator bandage securing a flexible sheath;

FIG. 7 is a top perspective view of a securement bandage assembly;

FIG. 8 is a top perspective view of an optional embodiment of a securement bandage assembly provided without a slit;

FIG. 9 is a breakaway perspective view of a securement bandage assembly;

FIG. 10 is a front view of an adhesion platform with wings and a center opening;

FIG. 11 is a perspective view of a flexible sheath having a distal narrowed catheter end; and

FIG. 12 is a perspective view of a flexible sheath through the center opening of adhesion platform

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

### DETAILED DESCRIPTIONS OF THE DISCLOSURE

In one embodiment, the present invention is a securement bandage for ensuring fluid transport to and/or from a patient. In alternate embodiments, the present invention may be a securement bandage assembly for securing a flexible sheath to a patient or a kit for ensuring fluid transport to and/or from a patient. The present invention may be used on multiple body surfaces. The principal concept behind the present invention is to provide a means for a user, such as a doctor or nurse, to effectively secure a flexible sheath, which may be a tube or catheter, to a patient.

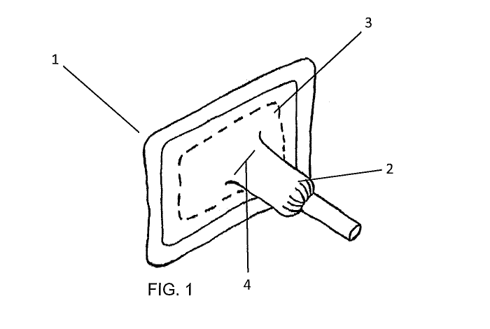

In a preferred embodiment as illustrated in FIG. 1, a securement bandage 1 for ensuring fluid transport to or from a patient is provided. The securement bandage comprises a flexible sheath 2 having a base end opening with a distal catheter end and an integrated flange adhesion platform 3 located at a base end of the flexible sheath extending from the base end opening. The integrated flange adhesion platform is provided with a center opening 4 to negotiate an extension from the body, such as a penis, nose or ear. The flexible sheath may itself be a condom catheter and is inserted or placed on a patient. The integrated flange adhesion platform is applied to the skin to keep the flexible sheath immobile. This securement bandage is most effective when fluids are being transported outside and away from the body for collection. As shown in FIG. 1, where the integrated flange adhesion platform embodies a rectangular shape, the integrated flange adhesion platform may comprise greater surface areas of adhesion on at least two adjacent sides relative to an opposing side of lesser surface area of adhesion. Most preferably, the integrated flange adhesion platform is made of substantially the same material as the flexible sheath, such as polyurethane or analogous material. The material could also be acrylic based, rubber based, silicone based, and polyurethane based. The securement bandage includes an adhesive. The adhesive could be acrylic based, rubber based, silicone based, and polyurethane based adhesives.

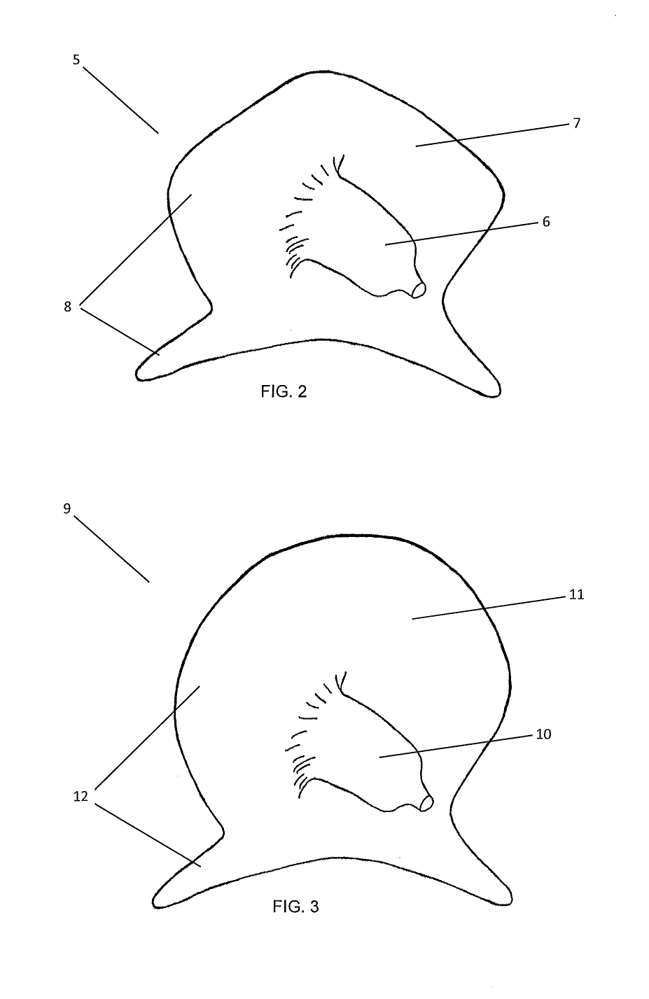

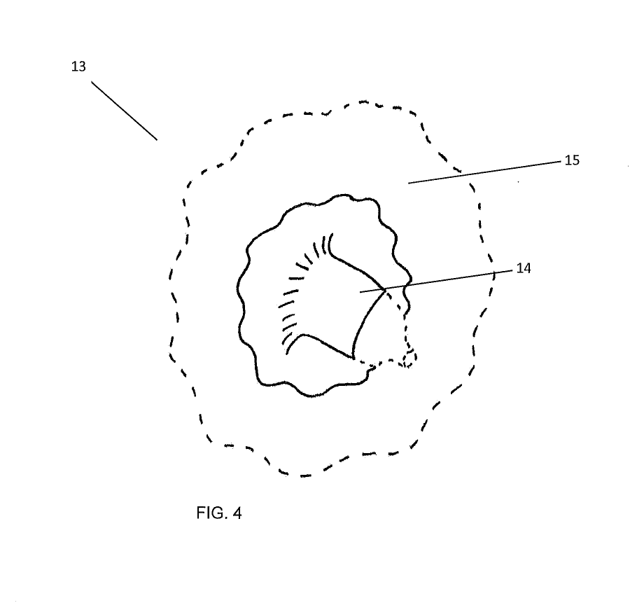

Now referring to FIGS. 2, 3, and 4, the securement bandage 5, 9 and 13 may be manufactured in various shapes or embodiments to negotiate various body parts, including limbs such as legs, rounded body parts such as cheeks, or other similar body parts. FIGS. 2 and 3 illustrate examples of shapes for these applications. In these designs, the integrated flange adhesion platform 7, 11 and 15 may be provided with wings 8 and 12 to aid in negotiating the above mentioned body parts and providing additional adhesion surface area. It is most preferred to provide the integrated flange adhesion platform with a large surface area of adhesion above and immediately adjacent to the flexible sheath. Less surface area of adhesion is required below the flexible sheath since the weight of the flexible sheath 6, 10 and 14 needs to be supported from above. Therefore, a substantially diamond shaped integrated flange adhesive platform 7 as shown in FIG. 2 or a substantially circular shaped integrated flange adhesive platform 11 as shown in FIG. 3 may be most optimal. However, these designs are meant to be illustrative and not limiting. FIG. 4 illustrates the securement bandage 13 without a particular shape design for the integrated flange adhesion platform 13 since it is envisioned that the shape of the adhesion platform may be altered to accommodate alternate body parts. The shape for the adhesion platform could be altered by cutting the adhesion platform to the desired shape.

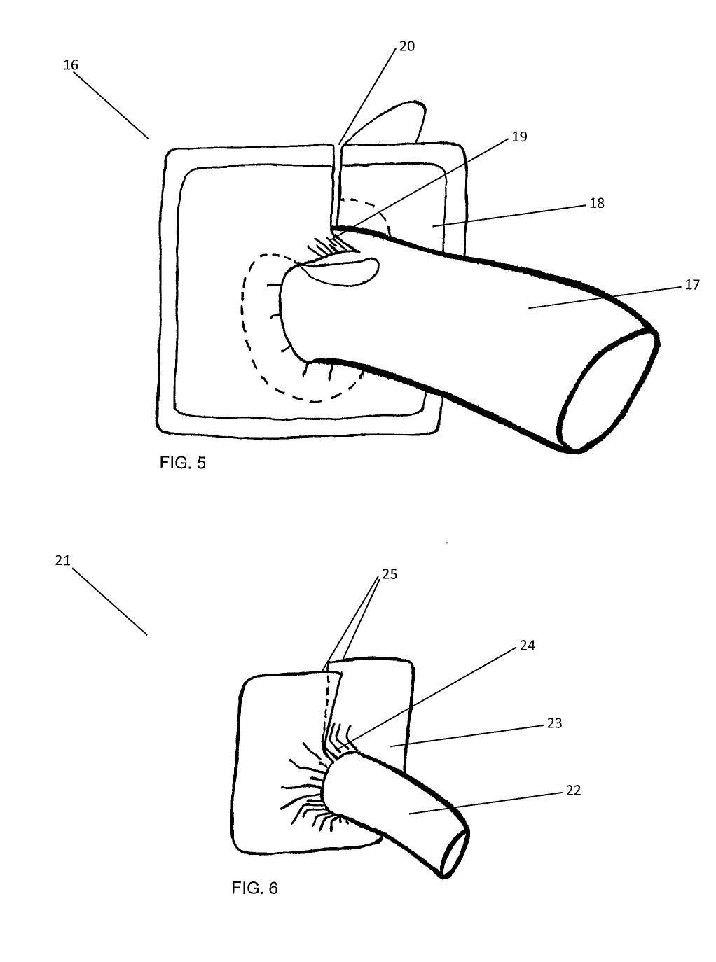

In another embodiment as illustrated in FIGS. 5 and 6, a securement bandage assembly 16, 21 for securing a flexible sheath to a patient is provided. The flexible sheath 17, 22 may be a tube or catheter secured by the sheath adhesion area 19, 24. In this embodiment, the securement bandage assembly may be manufactured and sold separately from flexible sheath. In this design, the securement bandage assembly is preferably provided with a slit 20 formed in a part of the adhesive applicator bandage 18, 23 to allow for easier securement by the user. The slit provides utility if the flexible sheath has already been positioned on the patient and cannot be removed. An additional advantage of this design is that flaps 25 are created by the slit, which may overlap when the securement bandage assembly is applied to the patient, creating additional surface area other than the patient's skin for the securement bandage assembly to adhere to. This concept is specifically illustrated in FIG. 6. Because skin is porous and perspires, adhesion of the securement bandage assembly to itself provides an advantage in ensuring lasting adhesion of the securement bandage assembly to the patient.

Alternatively, as shown in FIG. 8, the securement bandage assembly may not be provided with a slit. This design is envisioned to function well for flexible sheaths that are not yet attached to drainage tubes, such as short catheters (not shown).

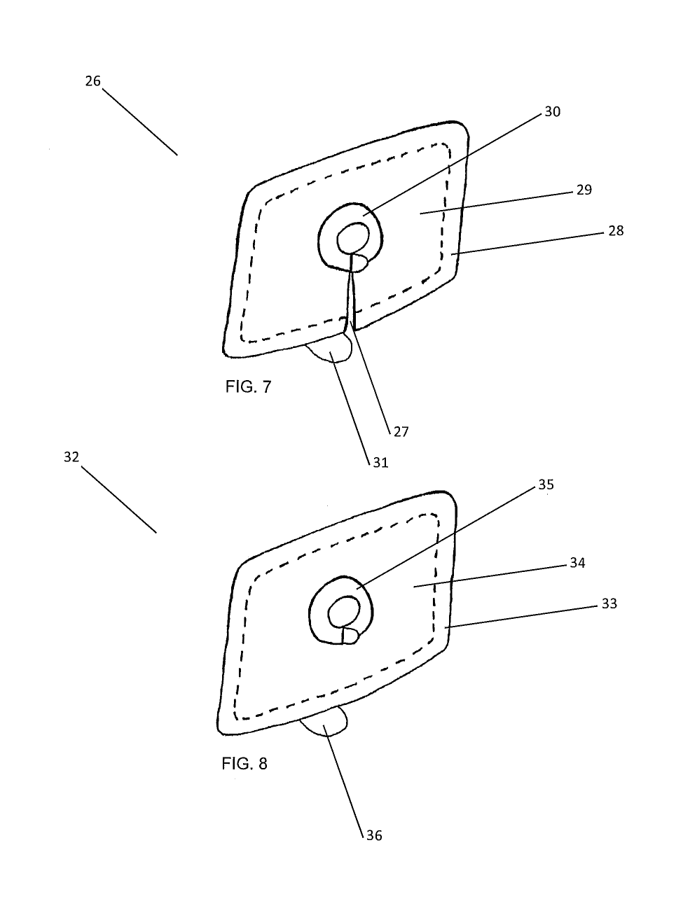

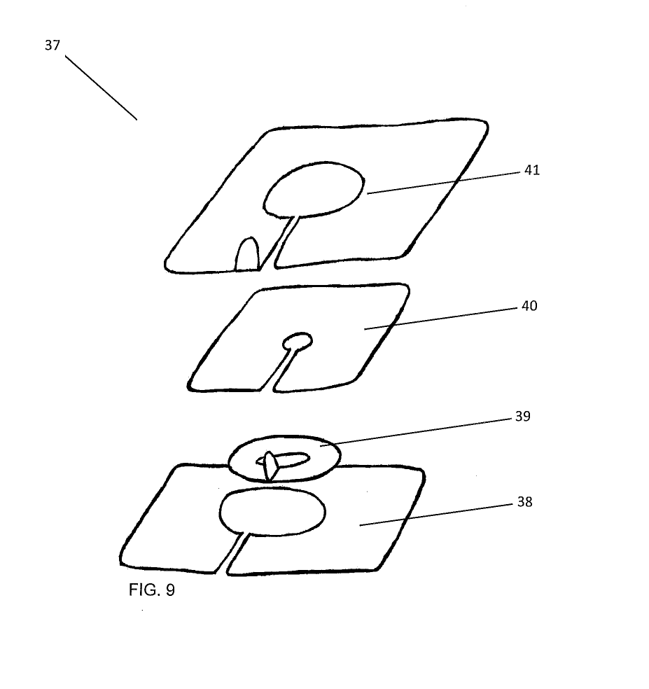

Now referring to FIGS. 7, 8, and 9, the securement bandage assembly 26, 32 and 37 is provided with an adhesive applicator bandage 29, 34 and 40, a frame 28, 33 and 41, a bandage backing 38, and a sheath backing 39. In some embodiments, a slit 27 is included in the assembly. The adhesive applicator bandage is provided with a center opening for negotiating the flexible sheath. The frame supports the adhesive applicator bandage, thereby maintaining the general integrity of the adhesive applicator bandage and preventing it from collapsing. The frame is preferably located on a front end of the adhesive applicator bandage and is shaped to cover an internal perimeter of the adhesive applicator bandage. Although a rectangular shape is illustrated in FIGS. 5 and 6, the more optimal shape is generally in the form of FIG. 2 or 3 with a slit above or below the center opening for attachment. The more optimal shape is provided with a greater surface area for adhesion just above the center opening to provide for sufficient attachment of the flexible sheath. The frame is preferably provided with a means of removal from the adhesive applicator bandage so that little or no adhesive residue is left on the front end of the adhesive applicator bandage after removal of the frame. The frame may be provided with a tongue or tab 31, 36 to allow the user to more easily remove the frame from the adhesive applicator bandage when needed.

The sheath backing 39 is located on a back end of the adhesive applicator bandage 40 to protect a sheath adhesive area of the adhesive applicator bandage. The sheath adhesive area 30, 35 is used to secure a flexible sheath to the adhesive applicator bandage before the secured pieces are collectively placed on the patient. The sheath backing presides substantially tangential of the center opening and is of sufficient width to provide an adhesive seal between the adhesive applicator bandage and the flexible sheath. Most preferably, the flexible sheath is a condom catheter. In this embodiment, the sheath backing should be optimally designed to conform to a protrusion created by the condom catheter. The sheath backing is ideally removed first and the sheath adhesive area of the adhesive applicator bandage is then applied to a base of the condom catheter or an outer wall of the condom catheter located near the base.

Once the adhesive applicator bandage is secured to the flexible sheath, the bandage backing is removed just prior to adhering the securement bandage assembly to the patient. The bandage backing presides radially external to the sheath backing so that the bandage backing covers substantially the portion of the adhesive applicator bandage intended to be secured to the patient. After the bandage backing is removed, the adhesive applicator bandage is applied to the patient, thereby securing the securement bandage assembly: The frame may be left attached to the securement bandage assembly; however, most users will be inclined to remove the frame so that the securement bandage assembly will better conform to the patient's body.

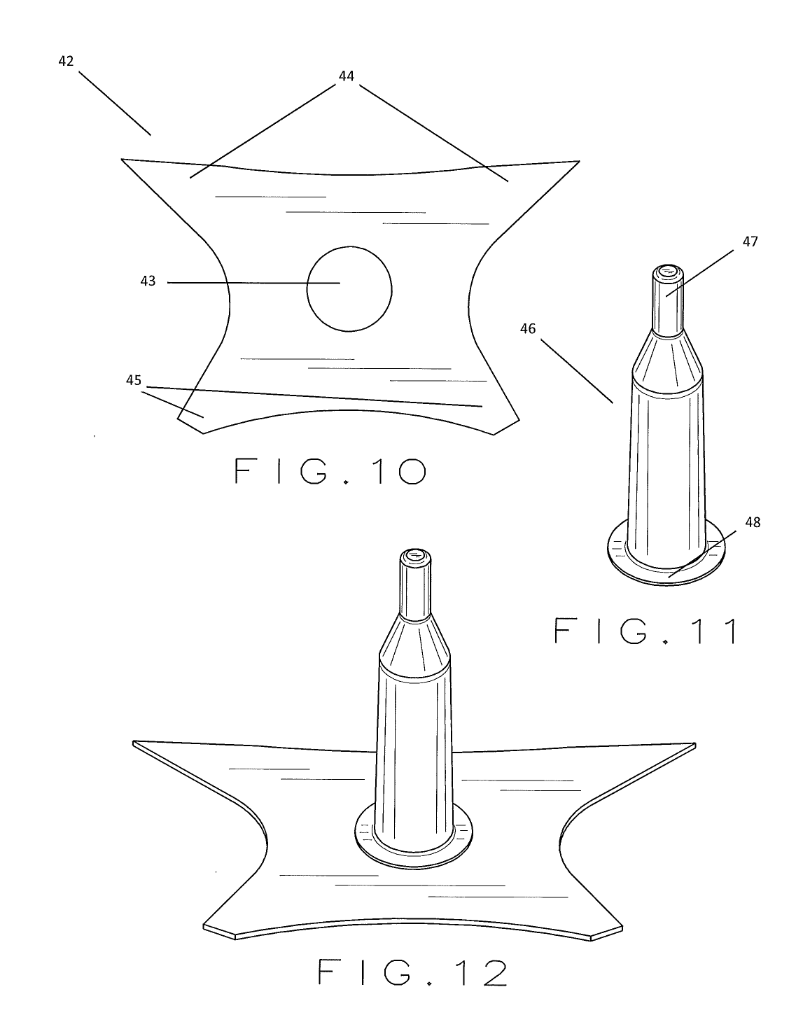

In another embodiment illustrated in FIGS. 10, 11 and 12, an adhesion platform 42 with a center opening 43 and a winged configuration, wherein the top pair of wings 44 is larger than the bottom pair of wings 45, is joined with an elastomeric sheath 46, wherein the elastomeric sheath fits through the center opening of the adhesion platform. The elastomeric sheath is a hollow tube with a distal narrowed catheter end 47. At the base of the elastomeric sheath is a circular base flange 48 that exceeds the diameter of the center opening. Thus, the elastomeric sheath can fit through the center opening of the adhesion platform by the distal narrowed catheter end, but the circular base flange with a larger diameter than center opening can resist passage there through. The circular base flange is preferably heat sealed to the adhesion platform. Optionally, in another embodiment not shown, the circular base flange may be secured to the adhesion platform using the adhesion of the adhesion platform by placing the adhesion platform over the circular base flange and adhering the adhesion platform to the patient and circular base flange. Still further in yet another embodiment, the circular base flange can have an adhesive on its top side for adhesively attaching to the adhesion platform and/or adhesive on its bottom side for adhesively attaching to the patient. The geometry of the flange can be other than round so long as it does not allow passage through the center opening without tearing.

In an alternate embodiment, a kit for ensuring fluid transport to or from a patient, the kit comprising a securement bandage.

In an alternate embodiment, a kit for ensuring fluid transport to or from a patient is provided. The kit comprises a flexible sheath and a securement bandage assembly adhesively connectable to the flexible sheath. The flexible sheath may be a tube or catheter, and the securement bandage assembly may be embodied as described above.

Another embodiment is a method of assemblying a securement bandage comprising forming a flexible sheath having a base end opening with a circular base flange extending from and about the opening and further having a distal narrowed catheter end; forming an adhesion platform having a center opening communicable with the base end opening and adhesively attached thereto; and attaching the flexible sheath to the adhesion platform, wherein the distal narrowed catheter end enters through the opening and the circular base flange adheres tangentially to the opening.

While several particular embodiments of the present invention have been described herein, it will be appreciated by those skilled in the art that changes and modifications may be made thereto without departing from the invention in its broader aspects and as set forth in the following claims.

Condom Cather Design Features

The sheath can be molded and the adhesion platform or the adhesive applicator bandage can be dye cut. Both parts can then be heat sealed together prior to sterilization and packaging. Thus, the end user can have the integrated device to be placed on the patient.

For the adhesive platform or the adhesive applicator bandage, the specific description of the material is 4.0 mil [0.1 mm] Medical Grade Silicone Adhesive.

The backing of the adhesive surface can have scores (cuts along which the base flaps can separate) going 12 o'clock to 6 o'clock and 3 o'clock to 9 o'clock, so there will be four quadrant flaps.

The sheath portion can be made of polyurethane resin. It can also be acrylic based, rubber based, silicone based, or polyurethane based.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Other aspects, objects and advantages of the present disclosure can be obtained from a study of the drawings, the disclosure and the appended claim.

1. A securement bandage for ensuring fluid transport to or from a patient, the securement bandage comprising:
a flexible sheath having a base end opening and a distal catheter end, and an integrated flange adhesion platform extending from the base end opening of the flexible sheath, wherein the integrated flange adhesion platform is provided with a center opening communicable with the base end opening.

2. The securement bandage of claim 1, wherein the flexible sheath of claim 1 is a condom catheter.

3. The securement bandage of claim 1, wherein the integrated flange adhesion platform has a perimeter geometry selected from the group consisting of circular, triangular, rectangular, square, and other closed geometry.

4. The securement bandage of claim 1, wherein the integrated flange adhesion platform comprises greater surface areas of adhesion on at least two adjacent sides relative to an opposing side of lesser surface area of adhesion.

5. The securement bandage of claim 1, wherein the integrated flange adhesion platform has a larger surface area of adhesion above and immediately adjacent to the flexible sheath relative to the surface area below the flexible sheath.

6. The securement bandage of claim 1, wherein the integrated flange adhesion platform can be altered in its shape.

7. The securement bandage of claim 6, wherein the integrated flange adhesion platform can be cut into a desired shape.

8. The securement bandage of claim 1, wherein the integrated flange adhesion platform is made of substantially the same material as the flexible sheath.

9. The securement bandage of claim 8, wherein the material is polyurethane.

10. The securement bandage of claim 1, wherein the integrated flange adhesion platform is made of a material selected from a group consisting of acrylic based, rubber based, silicone based, polyurethane based, and a combination thereof.

11. The securement bandage of claim 1, comprising an adhesive.

12. The securement bandage of claim 11, wherein the adhesive is selected from a group consisting of acrylic based, rubber based, silicone based, polyurethane based adhesives and a combination thereof.

13. The securement bandage of claim 1, wherein the integrated flange adhesion platform has winged appendages.

14. A securement bandage assembly for securing a flexible sheath to a patient comprising:
an adhesive applicator bandage with a center opening, and
a flexible sheath adhesive area around the center opening.

15. The securement bandage assembly of claim 14, wherein the adhesive applicator bandage has a slit, wherein flaps may overlap creating additional surface area other than the patient's skin for the securement bandage assembly to adhere.

16. The securement bandage assembly of claim 14 comprising:
a frame located on a front end of the adhesive applicator bandage;
a bandage backing and a sheath backing located on a back end of the adhesive applicator bandage, wherein the sheath backing presides substantially tangential of the center opening and the bandage backing presides radially external to the sheath backing.

17. A securement bandage assembly comprising:
an adhesive applicator bandage having a center opening and an adhesive surface about the perimeter of the opening; and
a flexible, hollow sheath having a base end opening adhesively attached to the center opening and a distal catheter end.

18. The securement bandage assembly of claim 17, wherein the adhesive applicator bandage has a winged configuration.

19. The securement bandage assembly of claim 18, wherein the winged configuration has a top pair of wings larger than a bottom pair of wings.

20. An apparatus comprising:
a flexible sheath having a base end opening with a circular base flange extending from and about the opening and further having a distal narrowed catheter end; and
an adhesion platform having a center opening communicable with the base end opening and adhesively attached thereto.

21. The apparatus of claim 20, wherein the adhesion platform has a winged configuration.

22. A kit for ensuring fluid transport to or from a patient, the kit comprising:
a flexible sheath, and
a securement bandage assembly adhesively connectable to the flexible sheath.

23. A method of assemblying a securement bandage comprising:
forming a flexible sheath having a base end opening with a circular base flange extending from and about the opening and further having a distal narrowed catheter end;
forming an adhesion platform having a center opening communicable with the base end opening;
attaching the flexible sheath to the adhesion platform, wherein the distal narrowed catheter end enters through the opening and the circular base flange adheres tangentially to the opening; and
attaching the adhesion platform to a patient.
